(19) United States
(12) Patent Application Publication
(10) Pub. No.: US 2012/0289874 A1
(43) Pub. Date: Nov. 15, 2012

(54) Spinal Posture Brace

(76) Inventor: David Eugene Nelson, Litchfield, MN (US)

(21) Appl. No.: 13/106,784

(22) Filed: May 12, 2011

(51) Int. Cl. A61F 5/01

(52) U.S. Cl. 602/19

(57) ABSTRACT

A spinal posture brace having a pair of assemblies, each including main support bar (2) with main support bar extension (3) and adjustment mechanism (4) at its inferior end and superior bar (1) and adjusting mechanism (4) at its superior end, with the assemblies being connected by thoracic strap (7), posterior waist strap (8), and anterior waste strap (9). Adjusting buckle (6) is present on each strap to adjust strap length. Lumbar strap (10) is present to provide additional support to the spine. Superior bar extension (11) is present to allow for height adjustment of superior bar (1).

### FEDERALLY SPONSORED RESEARCH

Not Applicable

### SEQUENCE LISTING OR PROGRAM

Not Applicable

### BACKGROUND

1. Field of Invention

This invention relates to braces for the human spine, specifically those that help promote proper posture and alignment and reduce the risk of injury.

2. Description of Prior Art

For years brace manufactures have been attempting to develop a back support that helps decrease the risk of injury to the back while a person is performing various daily activities at work and home. Some of these back supports are intended to protect the back during the healing process after a surgical procedure. Other back supports are intend to prevent injury and protect the backs of relatively healthy people, the backs of people who have had a history of back ailments, or the backs of people who have healed from a surgery. One of the main goals of preventing back injuries is to maintain the spine in a relatively neutral position and prevent movements that can increase the potential for injury, such as forward flexion and/or forward flexion with rotation. While some back supports can prevent unhealthy movements and stresses to back structures and others can maintain comfort and functionality for the user, the challenge has been to design a back support than can do both of these things effectively.

Many back supports, such as U.S. Pat. Nos. 5,302,171, 7,449,006, 5,833,638, and 5,399,150 involve some degree of strapping of various widths that encircle the torso of the user and have a securing means. Some of these supports, such as U.S. Pat. Nos. 5,040,524, 5,188,586, 5,349,706, and 5,503,620 have suspender type straps that connect to the brace and extend up over the shoulders of the user to help keep the brace from sliding down on the user. Some of these supports, such as U.S. Pat. Nos. 5,195,948, 5,984,885, 4,080,962 and 6,962,572 may include some type of flexible to semi-rigid support means typically positioned relatively parallel to the spine, although some have a more perpendicular orientation to the spine, in an attempt to give the back added support. U.S. Pat. No. 5,860,944 attempts to secure this type of device through attachments to the heels of the user to help to keep it in position. Although these supports are able to provide some degree of compression and warmth around back structures and potentially increase intra-abdominal pressure in an attempt to support the spine, their design limitations make them unable to actually prevent harmful movements and stresses to back structures such as forward bending and/or forward bending with rotation. With all of these supports, no matter how much you attempt to tighten them and secure them, the most potentially harmful back motions of forward bending and/or forward bending with rotation can still occur with relatively little restriction. This is due to the fact that no design elements of these supports are able to function in a manner to prevent these motions.

Other back supports are designed with more of a rigid material as a support means in an attempt to truly limit the unhealthy motions of the spine that can result in injury. U.S. Pat. Nos. 5,437,614, 5,259,831, 6,102,879, and 7,651,163 are made of a more rigid, molded material like plastic and are able to prevent unwanted movements of spinal structures. However, because of their design and structure, they are impractical for use during typical daily activities as they are generally not comfortable for the user. Their rigid material contours the body closely which can result in edges of the support that push into the body and are uncomfortable for the user. Because of the body surface area they cover and the type of material they are made of, these supports tend to hold in heat and perspiration which, again, makes them uncomfortable for the user. These types of braces are typically designed for, and used during, the healing process of more severe injuries and/or following surgical procedures to significantly limit movement. They are not designed to be used by the typical person during performance of daily activities.

U.S. Pat. Nos. 4,750,480, 4,871,998, 4,914,423, and 5,749,838 are supports that have some type of alarm system to alert the wearer that they are not adhering to proper spinal movement while performing various activities. They do not incorporate design elements that would provide any significant support or limitation of movement to prevent injury.

U.S. Pat. D604,856 and D616,553 are adhesive type supports that would directly adhere to the user. Their design and function does not incorporate any significant ability to limit movement to prevent injury. Another limitation with use of these adhesive supports is the difficulty for a user to apply these independently, as it is very difficult for most people to reach behind their back to apply them.

U.S. Pat. Nos. 5,120,288 and 5,067,484 are designed to affect the mid-back area of the user and have no ability to prevent undesirable motions through the spine as a user performs various daily activities. Instead, these supports are designed more for postural retraining purposes, using weights in an attempt to encourage proper posture.

U.S. Pat. No. 5,586,561 is designed to protect the spine during impact. It does not have design elements that would have any significant ability to help maintain the spine in a relatively neutral position and/or prevent undesirable spinal motion.

U.S. Pat. No. 5,713,840 is designed to provide a suspended lumbar spine support to assist with proper posture and support in a seated position, but has no ability to affect spine posture and prevent unhealthy movements in a standing position.

U.S. Pat. Nos. 4,773,106, 5,235,714, and 5,643,184 are designed to provide support to the back in a seated position, but have no ability to be utilized in a standing position while performing various daily and work related activities.

U.S. Pat. Nos. 6,190,340, 7,578,798, 6,991,611, and 5,860,896 are designed to provide support and improve posture through the thoracic spine area, but have no ability to affect the lumbar spine region.

U.S. Pat. Nos. 5,135,470 and 5,259,833 are designed with various flexible straps secured over the shoulders and around the waist of the user. Although these supports have some ability to limit unhealthy spinal movements, they are limited in their ability to be anchored effectively to fully prevent these movements. As a user attempts to bend forward at the spine, the securing strap around the waist is pulled up the torso of the user which results in the user still being able to perform the unhealthy movements of forward bending and/or forward bending with rotation. One of the supports attempts to secure the waist strap to the pants of the user. The pants of a user would typically not be secure enough to prevent the waist strap from being pulled up the torso of the user. Secondarily, as force is applied to the groin area of the user through the pants, through their attachment to the waist strap, this would result in discomfort for the user. Another of these types of supports, U.S. Pat. No. 5,816,251, attempts to secure the support device around the legs, but securing it in this manner results in significant difficulty trying to sit down or squat and increases leg discomfort when attempting to get into these positions.

U.S. Pat. Nos. 4,026,547 and 5,038,760 have securing straps that extend around the groin of the user which would make them uncomfortable for the user to wear throughout the day due to rubbing and pressure occurring in the groin area.

Several devices attempt to utilize firm rods or bars to provide support and restrict movement of the spine. Some of these devices, such as U.S. Pat. Nos. 7,150,719 and 6,280,405 are designed to allow the user to impart a traction type force to the spinal column while being somewhat mobile and are not truly designed to be utilized by a typical person to prevent injury while performing various daily activities. U.S. Pat. No. 7,150,719 has a securing means that comes across the chest of the user which can limit expansion of the chest resulting in some difficulty with breathing with heavier exertion. It also has a pelvic arch means extending across the pelvis and a superior bar extending behind the shoulders and neck of the user which make this brace impractical to use with performance of day-to-day strenuous activity in both a seated and standing position as the bars would get in the way of the user and potentially be uncomfortable. The device is designed to treat curvature issues of the spine, not to provide support and prevent injury during the performance of daily activity. Other such supports, such as U.S. Pat. Nos. 5,632,724, 6,280,405, and 5,199,940 have a firm rod or bar that extends along the anterior or poster aspect of the trunk of the user. While these devices are able to limit spinal movement the positioning of the bar results in difficulty sitting comfortably and also can result in uncomfortable pressure points for the user, especially as they attempt to move. U.S. Pat. Nos. 6,015,395 and 5,405,313 utilize pads that come under the arms to provide support to the spine, but putting pressure under the arm in this fashion compromises the neurovascular structures that run through this area which could be harmful to the user. Also, having pads under the arms in this fashion would be uncomfortable to use due to perspiration in this area with activity.

### OBJECTS AND ADVANTAGES

Several objects and advantages of the present invention are:
a) To provide a back support which prevents unhealthy motions of the lumbar and thoracic spine during performance of activities, especially forward bending and forward bending with rotation while, at the same time, allowing the user to perform other functional movements of the spine that are needed to perform daily activities;
b) To provide a back support which functions effectively and comfortably in a sitting or standing position;
c) To provide a back support which is easily adjustable to fit various sizes and shapes of users;
d) To provide a back support that is easy to put on and take off by the user;
e) To provide a back support that is comfortable for the user to wear even while performing strenuous activities.

Still further objects and advantages will become apparent from a consideration of the ensuing description and drawings.

### SUMMARY

In accordance with the present invention, a back support device compromises a pair of assemblies, each including a main support bar, a main support bar extension and associated adjustment mechanism at its inferior end, and a superior bar and associated adjusting mechanism at its superior end, with the assemblies being connected by a thoracic strap, a posterior waist strap, and an anterior waste strap. Adjusting buckles are present on each strap to adjust strap length. A lumbar strap is present to provide additional support to the spine.

### DRAWINGS

### Drawing Figures

In the drawings, closely related figures have the same number but different alphabetic suffixes.

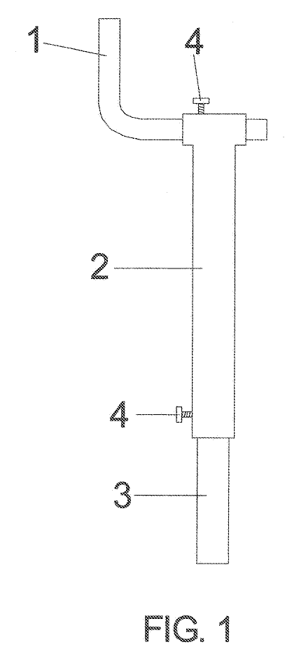

FIG. 1 illustrates a side view of the preferred embodiment of the present invention.

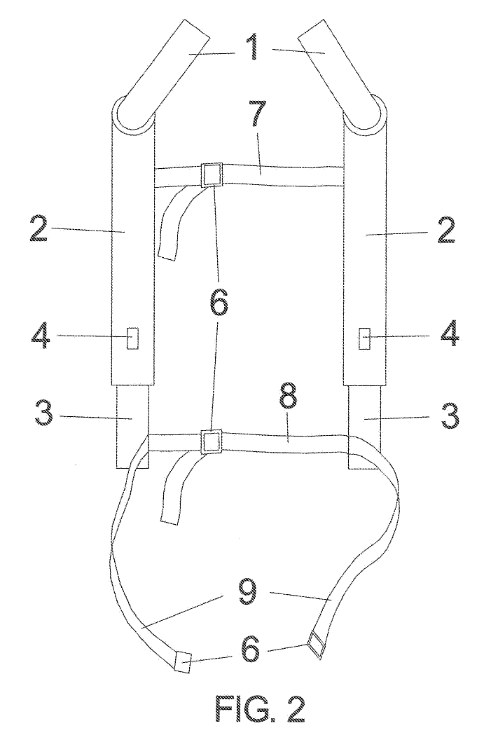

FIG. 2 illustrates a front view of the preferred embodiment of the present invention.

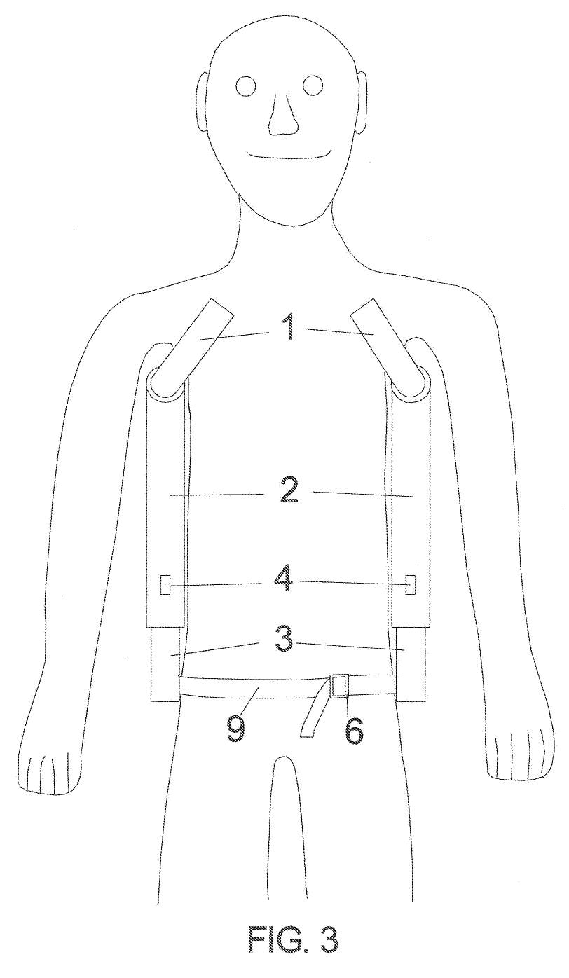

FIG. 3 illustrates a front view of the preferred embodiment of the present invention applied to a user.

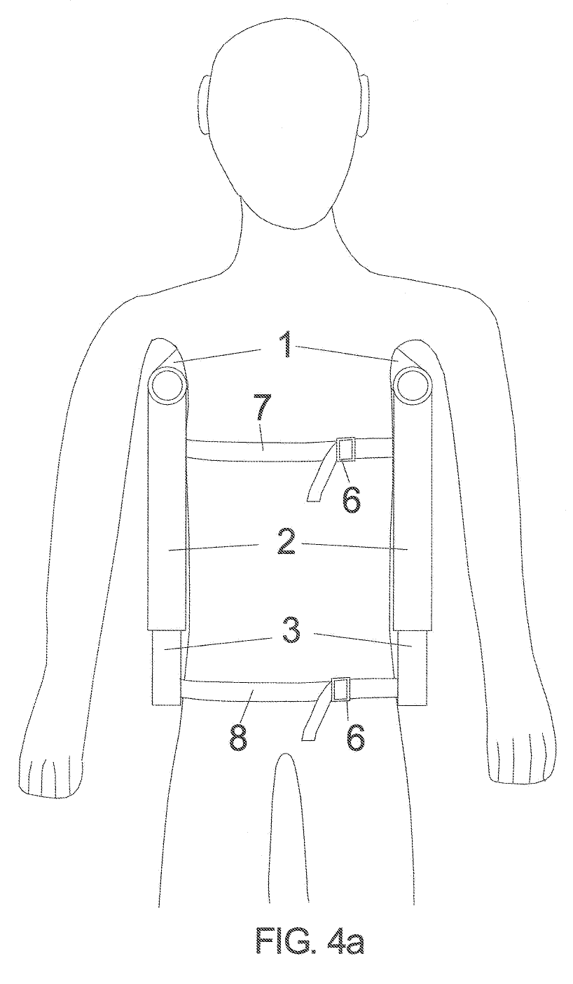

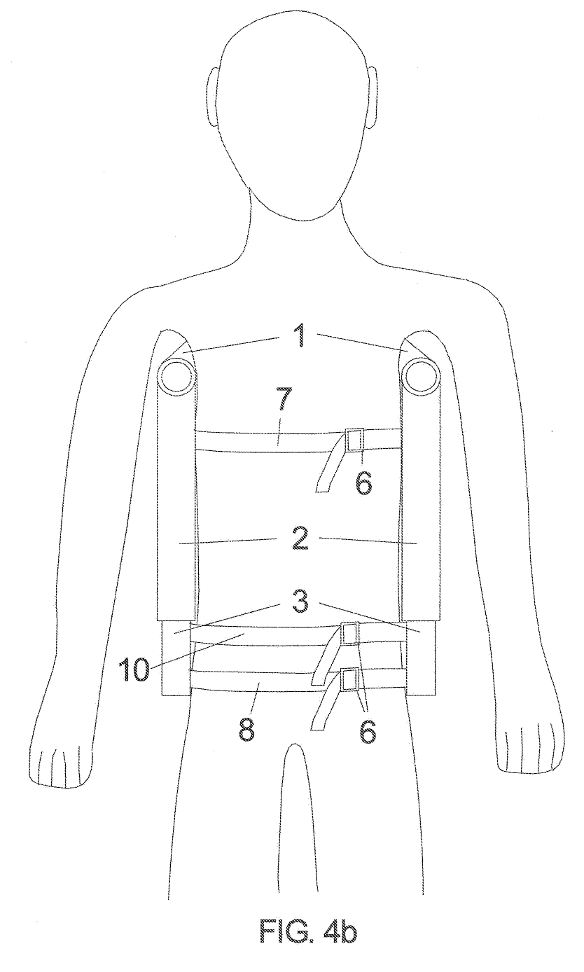

FIG. 4a illustrates a back view of the preferred embodiment of the present invention applied to a user.

FIG. 4b illustrates a back view of an alternate embodiment of the present invention applied to a user.

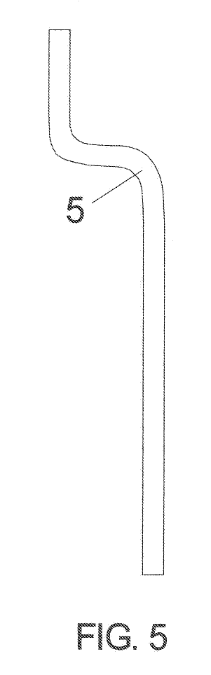

FIG. 5 illustrates a side view of an alternate embodiment of the present invention.

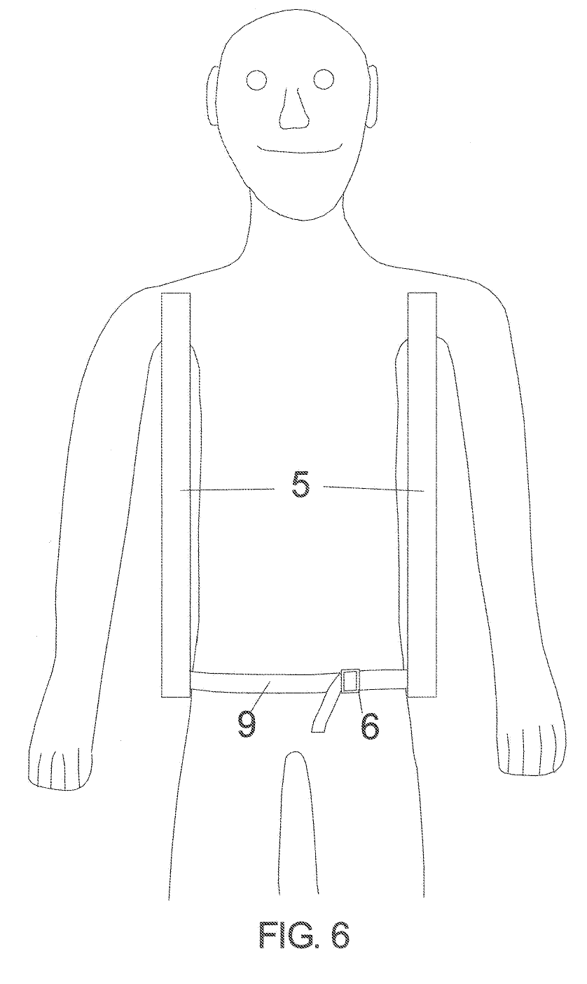

FIG. 6 illustrates a front view of an alternate embodiment of the present invention applied to a user.

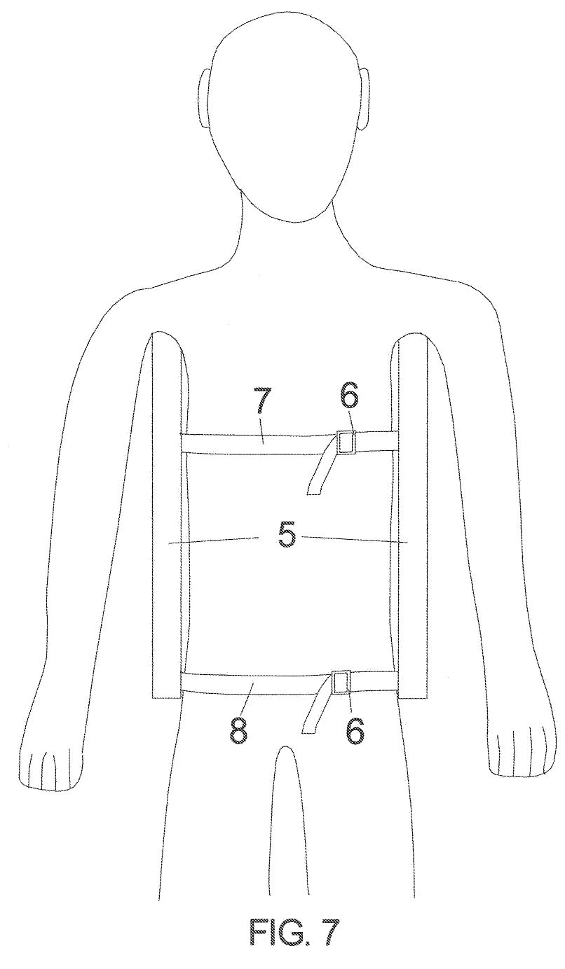

FIG. 7 illustrates a back view of an alternate embodiment of the present invention applied to a user.

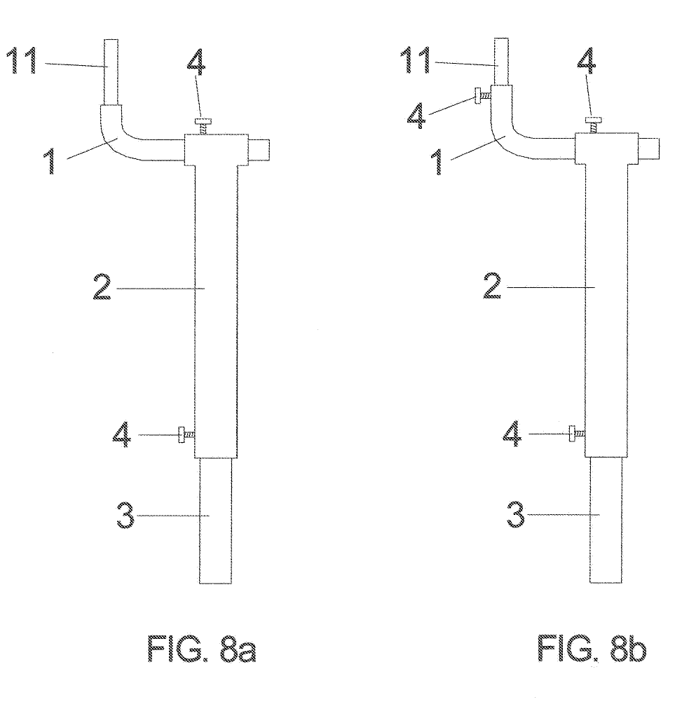

FIG. 8a illustrates a side view of an alternate embodiment of the present invention.

FIG. 8b illustrates a side view of an alternate embodiment of the present invention.

### REFERENCE NUMERALS IN DRAWINGS

1 Superior Bar
2 Main Support Bar
3 Main Support Bar Extension
4 Adjusting Mechanism
5 Standard Support Bar
6 Adjusting Buckle
7 Thoracic Strap
8 Posterior Waist Strap
9 Anterior Waist Strap
10 Lumbar Strap
11 Superior Bar Extension

### DETAILED DESCRIPTION

### Description

### FIGS. 1-4a—Preferred Embodiment

A preferred embodiment of the spinal posture brace is illustrated in FIG. 1-4a. FIG. 1 illustrates a side view of the spinal posture brace. The spinal posture brace includes two assemblies, each including a main support bar 2, a superior bar 1, a main support bar extension 3, and an adjusting mechanism 4. Said adjusting mechanism 4 could be a bolt, a screw, a toggle bolt, a push button mechanism, or any other type of mechanism that functions as outlined below. Said main support bar 2 has, at its superior end, an opening that passes horizontally through the bar allowing for said superior bar 1 to pass through it and move horizontally through this opening. The opening on the superior end of said main support bar 2 and the body of said superior bar 1 would be cylindrical in shape to allow said superior bar 1 to be able to rotate its position within the opening on the superior end of said main support bar 2. At its inferior end said main support bar 2 would be relatively hollow to allow said main support bar extension 3 to be inserted into said main support bar 2 or said main support bar extension 3 would be relatively hollow to allow said main support bar 2 to be inserted into said main support bar extension 3 to allow either of these components to move up and down the length of each other. Said adjusting mechanism 4 is present both at the superior and inferior ends of said main support bar 2 and is a mechanism that is able to contact said main support bar 2 and superior bar 1, at the superior end, and said main support bar 2 and main support bar extension 3, at the inferior end, to prevent movement of said superior bar 1 and said main support bar extension 3. Said adjusting mechanism 4 is able to be adjusted in such a manner as to contact or not contact said main support bar 2, said superior bar 1, and said main support bar extension 3 to prevent or allow movement respectively. Said superior bar 1 with its associated said adjusting mechanism 4 and said main support bar extension 3 with its associated said adjusting mechanism 4 work independently of each other. FIG. 2 illustrates a front view of the spinal posture brace. A thoracic strap 7 attaches to said main support bars 2 and has an adjusting buckle 6 which allows said thoracic strap 7 to be adjusted in length. A posterior waist strap 8 and an anterior waist strap 9 attach to said main support bar extensions 3, and each have said adjusting buckle 6 which allows said posterior waist strap 8 and said anterior waist strap 9 to be adjusted in length. FIG. 3 and FIG. 4a show the spinal posture brace applied to a user from both a front and back view, respectively. FIG. 3 and FIG. 4a will be described further in the operation section below.

### Operation

### FIGS. 1-4a—Preferred Embodiment

In the preferred embodiment of present invention, the spinal posture brace would first be positioned so that a said main support bar 2 was placed on the lateral aspect of each side of the user's torso, orientated parallel to the torso, with said thoracic strap 7 and said posterior waist strap 8 positioned along the back of the user as illustrated in FIG. 4a. Said main support bar extension 3 would then be moved up or down the length of main support bar 2 to achieve the desired length of the spinal posture brace. The corresponding said adjustment mechanism 4 would then be positioned to contact main support bar extension 3 to prevent any further movement. Said superior bar 1 would then be moved horizontally through the opening on the superior end of said main support bar 2 so that the front part of said superior bar 1 would contact the front torso or shoulder of the user. Said superior bar 1 would then be rotated within the superior end of said main support bar 2 so that it contacted the user in a spot deemed the most confortable by the user on the front torso or shoulder as illustrated in FIG. 3. After this the corresponding said adjustment mechanism 4 would be positioned to contact said superior bar 1 to prevent any further movement. Said thoracic strap 7, said posterior waist strap 8, and said anterior waist strap 9 would then be adjusted in length to secure the spinal posture brace to the user to maintain the brace in the above described position as illustrated in FIGS. 3 and 4a.

### Description

### FIGS. 5-7—Alternate Embodiment

An alternate embodiment of the spinal posture brace is illustrated in FIGS. 5-7. FIG. 5 displays a side view of a standard support bar 5 of the spinal posture brace. In this alternate embodiment the spinal posture brace includes two said standard support bars 5. As depicted in FIGS. 6 and 7 said thoracic strap 7, said posterior waist strap 8, and said anterior waist strap 9 attach to said standard support bars 5 with each strap having said adjusting buckle 6 which allows said thoracic strap 7, said posterior waist strap 8, and said anterior waist strap 9 to be adjusted in length. This alternate embodiment would not include said main support bar 2 and said superior bar 1, but it could include said main support bar extension 3 with its corresponding said adjusting mechanisms 4. Said main support bar extension 3 and its corresponding said adjusting mechanism 4 would function in the same manner as described in the preferred embodiment.

### Operation

### FIGS. 5-7—Alternate Embodiment

In an alternate embodiment of present invention, the spinal posture brace would first be positioned so that said standard support bar 5 was on the lateral aspect of each side of the user's torso, orientated parallel to the torso, with said thoracic strap 7 and said posterior waist strap 8 positioned along the back of the user as illustrated in FIG. 7. The superior aspect of said standard support bar 5 would contact the front torso or shoulder of the user. If said main support bar extension 3 was present with its corresponding said adjusting mechanism 4, this would then be adjusted to make the device the desired length. Said thoracic strap 7, said posterior waist strap 8, and said anterior waist strap 9 would then be adjusted in length to secure the spinal posture brace to the user to maintain the brace in the above described position as illustrated in FIGS. 6 and 7.

### Description

### FIG. 4b—Alternate Embodiment

An additional alternate embodiment of the spinal posture brace is illustrated in FIG. 4b but could be present in either the preferred embodiment of FIGS. 1-4a or in the alternate embodiment of FIGS. 5-7. This additional alternate embodiment includes a lumbar strap 10 which would include said adjusting buckle 6 to allow said lumbar strap 10 to be adjusted in length. If present in the preferred embodiment of FIGS. 1-4a, said lumbar strap 10 would be attached to said main support bar 2 or said main support bar extension 3. If present in the alternate embodiment of FIGS. 5-7, said lumbar strap 10 would be attached to said standard support bar 5.

### Operation

### FIG. 4b—Alternate Embodiment

In an additional alternate embodiment of the spinal posture brace, as illustrated in FIG. 4b, said lumbar strap 10 would positioned in the desired location and adjusted in length to contact the back of the user. This would provide additional support to the spine to prevent unwanted movements of spinal structures.

### Description

### FIG. 8a-8b—Alternate Embodiment

An additional alternate embodiment of the spinal posture brace is illustrated in FIG. 8a-8b but could be present in combination with any of the previously mentioned embodiments. This additional embodiment includes a superior bar extension 11 to be inserted into said superior bar 1 as illustrated in FIG. 8a or, as shown in FIG. 8b, said superior bar 1 being relatively hollow to allow said superior bar extension 11 to be inserted into said superior bar 1 or said superior bar extension 11 being relatively hollow to allow said superior bar 1 to be inserted into said superior bar extension 11 to enable either of these components to move up and down the length of each other along with said adjusting mechanism 4 being present at the anterior, superior end of superior bar 1. Said adjusting mechanism 4 functions in the same manner as described above in the preferred embodiment above. Said adjusting mechanism 4 is able to be adjusted in such a manner as to contact said superior bar 1 and said superior bar extension 11 to prevent movement or is able to not contact said superior bar 1 and said superior bar extension 11 to allow movement.

### Operation

### FIG. 8a-8b—Alternate Embodiment

In an additional alternate embodiment of the spinal posture brace, as illustrated in FIG. 8a, said superior bar extension 11 could be a variety of lengths and would be able to insert into said superior bar 1 to allow for the overall height of this assembly to be adjustable to properly fit various sizes of users. Alternately, as illustrated in FIG. 8b, said superior bar extension 11 could be a single length able to be inserted into, or over the top of, said superior bar 1 with said adjustment mechanism 4 present. Said superior bar extension 11 could be positioned to the correct height for the user and, after this, the corresponding said adjustment mechanism 4 would be positioned to contact said superior bar 1 and said superior bar extension 11 to prevent any further movement. This would also allow for the overall height of this assembly to be adjustable to properly fit various sizes of users.

### CONCLUSIONS, RAMIFICATIONS, AND SCOPE OF INVENTION

Thus the reader will see that the spinal posture brace of present invention provides a functional, versatile, and innovative approach to providing support to the spine of a human body to help promote proper posture and alignment and reduce the risk of injury. The device allows for the user to be able to perform functional movements while maintaining the spine in a relatively neutral position and preventing movements that can increase the potential for injury, such as forward flexion and/or forward flexion with rotation. The device is able to maintain comfort for the user during the performance of strenuous activity due to its adjustability, its application of contact and pressure to areas of the body that would be comfortable for the user, and its small area of body surface contact compared to other back support devices. The device is also able to accommodate numerous variations in user size and shape, can be effectively used in a seated or standing position, can easily be reapplied by an individual without external assistance, and is easily portable.

While my above options contain many specificities, these should not be construed as limitations on the scope of the invention, but rather as an exemplification of one preferred embodiment there of. Many other variations are possible. For example, straps could have various means of attachment to the device, a variety of adjusting buckle type devices could be utilized including Velcro, the various bars of the device could be differing shapes, the various bars could have differing combinations of being solid and hollow to allow them to slide up and down the length of each other, a means for securing the loose ends of straps to the device could be added, and a variety of modes of operation for the adjusting mechanisms are possible such as push button type mechanisms that would not change the overall form and function of my present invention.

Thus the scope of the invention should be determined by the appended claims and their legal equivalents, rather than by the examples given.

I claim:

1. A spinal posture brace providing support to the spine of a human body to help promote proper posture and alignment and reduce the risk of injury including: (a) a set of standard support bars (b) a plurality of straps for securing said standard support bars in position and limiting motion (c) a means for adjusting the length of said straps.

2. The spinal posture brace of claim 1, further including: (a) a means for adjusting the length of said standard support bars.

3. A spinal posture brace providing support to the spine of a human body to help promote proper posture and alignment and reduce the risk of injury including: (a) a set of main support bars (b) a plurality of straps for securing said main support bars in position and limiting motion (c) a means for adjusting the length of said straps (d) a means for adjusting the length of said main support bars (e) a superior bar.

4. The spinal posture brace of claim 3, further including: (a) a means for adjusting the position of said superior bar.

5. The spinal posture brace of claim 3, further including: (a) a means for adjusting the height of said superior bar.

6. The spinal posture brace of claim 3, further including: (a) a means for adjusting the position of said superior bar (b) a means for adjusting the height of said superior bar.
